(19) United States
(12) Patent Application Publication
(10) Pub. No.: US 2002/0182629 A1
(43) Pub. Date: Dec. 5, 2002

(54) Flexible substrate for microarray assays

(76) Inventor: Patrick M. Rich, Orchard Lake, MI (US)

Correspondence Address: Ronald W. Citkowski, Gifford, Krass, Groh, Sprinkle,, Anderson & Citkowski, P.C., 280 N. Old Woodward Ave., Suite 400, Birmingham, MI, 48009, US

(21) Appl. No.: 10/139,877

(22) Filed: May 7, 2002

Related U.S. Application Data
(60) Provisional application No. 60/294,788, filed on May 31, 2001.

(51) Int. Cl. C12Q 1/68; G01N 33/53; G01N 33/542; C12M 1/34

(52) U.S. Cl. 435/6; 435/7.9; 435/287.2

(57) ABSTRACT

A flexible microarray assay member includes a flexible substrate, such as a sheet of organic polymer, having an array of biomolecular samples affixed thereto by a linker material. The biomolecular samples may include nucleotides, proteins, antibodies and the like. The flexible array may be used in microchip assay technologies. It may also be used as a transfer agent for the manufacture of other arrays, as for example by an offset printing process.

### RELATED APPLICATION

This patent application claims priority of U.S. Provisional Patent Application No. 60/294,788 filed May 31, 2001, entitled “Flexible Substrate for DNA Microchips.”

### FIELD OF THE INVENTION

The invention relates to reaction supports and more particularly, to flexible supports for molecular interaction. Most specifically, the invention relates to microarrays, electrophoresis gels, and the like having flexible substrates.

### DESCRIPTION OF THE RELATED ART

Hybridization or binding of biomolecules such as DNA, RNA, peptides, polypeptides, proteins, antibodies, antigens, ligands and combinations of these to target molecules is an efficient and cost-effective way to screen for molecular interaction. For example, an array of oligonucleotides may be deposited and immobilized on a support which is then exposed to sample DNA or RNA under conditions which allow hybridization. Following removal of non-bound molecules, the identity or quantity of the bound molecules is determined. Very high density microarrays of biomolecules have been commercialized, and they allow for rapid screening a large number of molecular species.

Traditionally, the solid support used in such microarray assays is planar glass, modified or coated in a way so as to allow for the desired molecular binding to the support surface. Representative prior art disclosing rigid, solid supports includes: U.S. Pat. No. 5,445,934 that discloses a rigid or semi-rigid substrate having oligonucleotides covalently attached to the surface in discrete known regions; U.S. Pat. No. 5,919,523 that discloses a method of preparing a rigid or semi-rigid derivatized solid support which is linked to a polymer via functional groups on the derivatizing agent; and U.S. Pat. No. 5,959,098 that discloses methods for forming polymer arrays on a rigid or semi-rigid solid substrate wherein functional groups are protected with photolabile groups and are deprotectable independently based on location on the substrate.

Likewise, electrophoresis gels have previously employed rigid substrates such as glass substrates. Technological progress has now made it feasible to automate the production, processing and read-out of microarrays and electrophoresis gels. However, commonly used reaction supports, such as glass or hard plastic, have the disadvantage of being rigid or semi-rigid, making them susceptible to damage or breakage in the course of such automated processes. Thus, there is a need for flexible reaction supports for microarrays, microarray assays and electrophoresis gels.

### BRIEF DESCRIPTION OF THE INVENTION

There is disclosed herein a flexible microarray assay member. The assay member includes a flexible substrate having a linker material disposed upon at least a portion of a first surface thereof. The linker material is capable of bonding to a molecular species. A plurality of biomolecular samples, each member of said plurality being of different composition from the other members thereof, is bonded to the substrate through said linker material so that each member of said plurality is disposed in a different, discrete region of the substrate so as to provide an array of said samples.

In some embodiments, the linker material is an integral component of the substrate material, while in other instances, the linker material is separately applied thereto, in which instance, the linker material may be applied to the entirety of the substrate, or it may be applied to discrete portions thereof, as for example to portions corresponding to the regions in which the members of the array are bound. In yet other embodiments of the invention, an additional coupler material may be utilized to facilitate bonding of the biomolecules to the substrate. The coupler may be affixed to the linker, or to the molecules.

The substrate preferably comprises a flexible organic polymer; however, it may further include inorganic materials therein, such as reinforcement materials, fillers, stiffeners, and the like.

In accord with another aspect of the present invention, the flexible array may be employed as an intermediate transfer medium for use in the manufacture of microarray assays. In this regard, the flexible array may be employed in an offset printing process to transfer all, or a portion, of the array of material bonded thereto onto another support member which will receive and bind the molecular species thereto. In this manner, arrays can be created on substrates which may not be amenable to prior handling steps.

### DETAILED DESCRIPTION OF THE INVENTION

The present invention provides flexible solid supports for molecular interaction. The solid supports of the present invention are flexible; and in the context of this disclosure, flexible substrates are defined as being those which do not break or craze or become permanently deformed when bent through a plane of at least 15 degrees from planar.

Flexible solid supports of the type provided by the present invention are substrates for molecular interaction illustratively including polymer synthesis, oligonucleotide arrays, peptide arrays, ligand or receptor binding, antibody or antigen binding, electrophoresis and hybridization, all of which are described as microarray assays within the context of this disclosure. Examples of molecules that can be bound to the substrates of the present invention are nucleotides, amino acids, receptors, ligands, antibodies and antigens.

The flexible solid support is organic, inorganic, or a combination thereof. The inventive support takes any of various shapes including, fibers, particles, sheets, slides, spheres, and tubing. In a preferred embodiment, the flexible support has a planar surface. The inventive flexible support is optionally marked to indicate orientation. For example, a rectangular support may be marked to indicate its orientation by a cropped corner, a printed marking or the like. Other modifications to indicate orientation will be apparent to those skilled in the art.

While there is no practical limit on the size of substrates in accord with the present invention, limitations of typical assay procedures will require that most substrates have dimensions ranging from 1 millimeter² to 10 meters² and a thickness ranging from 1 micrometer to 20 millimeters.

The flexible solid support of the present invention includes a substratum having a linker attached to its surface. The linker serves to bind the subsequently applied molecular species to the substrate. In some instances the material of the substrate will function as the linker, in which case no further linker materials need by employed. In certain instances a coupler may also be employed to bind the molecular species to the linker, and this coupler may be bound to the linker, or to the molecular species.

Substrata

An inventive substratum is flexible and ranges in thickness from 1 micrometer to 20 millimeters. The substratum may be composed of any of a variety of materials, such as polymers, copolymers and mixtures of polymers, copolymers and inorganics that are flexible. Examples of suitable substratum materials are polyvinylfluoride, nitrocellulose, nylon, polycarbonate, polyester, polyethylene, (poly)ethylene naphthalate, poly (ethylene-co-4-methyl-1-pentene), polyimides, polypropylene, polysulfone, polystyrene, (poly)tetrafluoroethylene, (poly)vinylidendifluoride, epoxy resins or combinations thereof. In addition, substrata can be composites which include inorganic components such as glass fibers or particles, polygermanes, polystannanes, polyphosphazenes; silicates such as feldspar, talc, mica, wollastonite, clay, alkyl quaternary ammonium clay and fumed silica. Further suitable substrata will be readily apparent to one skilled in the art.

The substratum material of the support of the present invention is preferably optically transparent. Optionally, the substratum is coated on one side with a material with anti-reflective properties illustratively including a curable resin, a polymer such as Teflon and a layer of metal ions. The substrate, and any coating applied thereto, should have low autofluorescing properties so as to avoid interference and increase the optical clarity of fluorescent dyes used in analytical procedures.

Substratum Surface

The substratum surface will contain functional groups capable of interacting with the linker to form a bond. Such bonds are formed covalently, by charge interactions and by hydrogen bonding. Illustrative examples of functional groups include alkyl, Si—OH, carboxy, carbonyl, hydroxyl, amide, amine, amino, ether, ester, epoxides, cyanate, isocyanate, thiocyanate, sulfhydryl, disulfide, oxide, diazo, iodine, sulfonic or similar groups having chemical or potential chemical reactivity.

Linkers

The linker molecule has a first reactive moiety that attaches to a functional group on the surface of the substrate and a second reactive moiety that attaches to the molecular species to be bound to the substrate, or to a coupler which in turn binds, or is bound to, the molecular species. The first and second reactive moieties of the linker may be the same or different and illustratively include amide, amino, carboxy, epoxide, ester, hydroxy, isocyanate, isothiocyanate and thiol, as well as gels and agars. The linker will optionally have a protecting group attached to the second reactive moiety. The linker optionally binds to all available surface sites or is diluted so as to provide an even distribution of a desired number or density of available coupler binding sites. As noted above, the surface of the substrate may inherently be reactive so as to function as an integral linker. Once the desired number of linkers are bound to the surface, the remaining surface sites are blocked to prevent further reaction.

Where a pattern or defined distribution of binding sites is desired, the linker is applied to the substratum surface in the desired pattern or distribution for example by laser jet, by mechanical printing and by computer-assisted printing. Following linker binding, unreacted functional groups on the substratum surface are blocked to prevent further reaction.

Illustrative examples of linkers include silanes, aryl acetylene, diamines, diacids, polyalcohols, polyesters, polyethers, polylysine, polyarginine, polystyrene sulfonate, dextran sulfate, chondroitin, acrylamines, agars, polyacrylic acid, polymethacrylic acid, polyacrylamide, polyallylamine, maleic acid, substituted or unsubstituted polyalkylenes, polyamines, polyamides, polysufonates, polyoxides, polyalkyleneglycols, polystyrenic based polymers, polyacetals, polysaccharides, polycarbonates, polyurethanes, polyureas, polyamides, polyethyleneimines, polyarylene sulfides, polysiloxanes, polyimides, polyacetates, polymers of monoethylenically unsaturated monomers, polymers of polyvinylidene monomers and mixtures and copolymers of the above polymers. The choice of linker depends on the nature of the substrate's surface and the molecular species being bound thereto and selection of an appropriate combination will be evident to one skilled in the art. For example, where the surface has available Si—OH groups, appropriate linkers include aminoalkyltrialkoxysilanes, aminoalkyltrichlorosilanes, carboxyalkyltri-alkoxysilanes, epoxyalkyltrialkoxysilanes, hydroxyalkyltrialkoxysilanes and hydroxyalkyltrichlorosilanes. Further suitable silanes are listed in Silicon Compounds: Register & Review, from United Chemical Technologies, 5th Ed., 1991.

Couplers

The coupler has a first leaving group that attaches to a reactive moiety on the linker and a second leaving group that attaches to another molecule, such as a biomolecule. The first and second leaving groups may be the same or different and illustratively include epoxide, ester, isocyanate, isothiocyanate, carbonyl, hydroxyl, carboxyl, amine, thiol, thioester, thioether, phosphate, alkoxy, aryl, arylalkyl, sulfonamide and alkyl halide. The choice of coupler will depend on the linker used and the biomolecule to be bound and selection of an appropriate combination will be evident to one skilled in the art.

Illustrative examples of linkers include silanes, aryl acetylene, diamines, diacids, polyalcohols, polyesters, polyethers, polylysine, polyarginine, polystyrene sulfonate, dextran sulfate, chondroiting, polyacrylic acid, polymethacrylic acid, polyacrylamide, polyallylamine, maleic acid, substituted or unsubstituted polyalkylenes, polyamines, polyamides, polysufonates, polyoxides, polyalkyleneglycols, polystyrenic based polymers, polyacetals, polysaccharides, polycarbonates, polyurethanes, polyureas, polyamides, polyethyleneimines, polyarylene sulfides, polysiloxanes, polyimides, polyacetates, polymers of monoethylenically unsaturated monomers, polymers of polyvinylidene monomers and mixtures and copolymers of the above polymers. The choice of linker depends on the surface and coupler used and selection of an appropriate combination will be evident to one skilled in the art. For example, where the surface has available Si—OH groups, appropriate linkers include aminoalkyltrialkoxysilanes, aminoalkyltrichlorosilanes, carboxyalkyltrialkoxysilanes, epoxyalkyltrialkoxysilanes, hydroxyalkyltrialkoxysilanes and hydroxyalkyltrichlorosilanes.

Process for Making a Solid Support

The flexible support of the present invention is made by application of the linker to the substratum surface. The substratum surface may be derivatized to provide pendant reactive groups appropriate to bind to the chosen linker. The method of derivatization depends on the chosen substratum and linker and will be apparent to one skilled in the art. In other instances, the substratum requires no derivatization for use with the chosen linker. The linker is attached to the substratum by a method appropriate to the substratum-linker pair which will be apparent to one skilled in the art. The density of linker molecules on the substrate ranges from less than one uniform molecular layer to 50 molecular layers of the linker. The density of linker molecules attached to the substratum is modulated in any of a number of ways illustratively including, dilution of the linker and blocking of a portion of linker attachment sites. In a preferred embodiment, the linkers attached to the substratum in a predetermined pattern such that the linkers are located primarily on sub-regions of the substratum corresponding to the binding pattern of subsequently applied molecular species. A patterned distribution of linkers is achieved by any of several methods illustratively including masking and blocking sites where linker binding is not desirable and then allowing the linkers to come in contact with the substratum. Optionally, the linker is directly applied to the substratum in the desired pattern by a method illustratively including laser printing, ink jet printing, pin printing, quill printing, and combinations thereof.

In those instances where the substrate further includes a separate coupler, that coupler is subsequently attached to the linker. The coupler has an appropriate reactive moiety available for attachment to the linker and an appropriate reactive moiety for attachment to a molecular species. The density of coupler sites will depend on the density of available linker sites. In a preferred embodiment, linker sites are exposed to a concentration of coupler such that substantially all available linker sites bind coupler. In another embodiment, the density of coupler sites is controlled by limiting the amount of coupler in contact with linker sites, for example, by coupler dilution. Optionally, the coupler is applied to the linker in a desired pattern by a method illustratively including laser printing, ink jet printing, pin printing, quill printing, and combinations thereof.

In another embodiment of the present invention, the linker and coupler are attached to each other in a first step and the complex of linker and coupler is then attached to the substrate. The application of the linker and coupler complex in a desired pattern is achieved by a method illustratively including laser printing, ink jet printing, pin printing, quill printing, and combinations thereof.

The inventive flexible support is optionally marked for identification purposes. The mark takes any of various forms known illustratively including a number, pattern or code, such as a bar code. The appropriate technique for applying the mark to the flexible support will be apparent to one skilled in the art and such techniques illustratively include laser printing, ink jet printing, pin printing, quill printing, thermal transfer and combinations thereof.

Use of the Flexible Substrate as a Transfer Medium

In another aspect of this invention, the flexible substrate is used as a medium for transfer of a component to a second support which may be a rigid or a flexible support. Such transfer may be implemented, for example by offset printing means so as to apply a pattern of material to a substrate not amenable to the printing process due to its rigidity. For example, an oligonucleotide bound to an inventive flexible support is transferred to a suitable rigid substrate such as glass containing a binding agent such as a pendant amine or aldehyde functional group, by bringing the oligonucleotide and the functional group into proximity under conditions suitable for transfer.

In another embodiment, a linker coupler combination or a coupler alone, is transferred to a suitable rigid substrate by bringing the coupler into proximity with a pendant functional group on the rigid substrate under conditions suitable for transfer.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A flexible microarray assay member comprising:
a flexible substrate;
a linker material disposed upon at least a portion of a first surface of said flexible substrate, said linker material being capable of bonding to a molecular species; and
a plurality of biomolecular samples, each sample of said plurality being of different composition from the other members of said plurality, and each sample being bound to a different region of said first surface of said substrate through said linker material.

2. The assay member of claim 1, wherein said biomolecular samples are selected from the group consisting of: DNA, RNA, oligonucleotides, peptides, polypeptides, proteins, antibodies, antigens, ligands, and combinations thereof.

3. The assay member of claim 1, wherein said substrate comprises a polymeric material.

4. The assay member of claim 3, wherein said polymer is selected from the group consisting of: polyvinylflouride, nitrocellulose, nylon, polyester polycarbonate, polyethylene, (poly)ethylene naphthalate, poly (ethylene-co-4-methyl-1-pentene), polyimides, polypropylene, polysulfone, polystyrene, (poly)tetrafluoroethylene, (poly)vinylidendifluoride and epoxy resin.

5. The assay member of claim 3, wherein said organic polymer further includes an inorganic material.

6. The assay member of claim 1, wherein said linker material directly binds said biomolecular samples to said substrate.

7. The assay member of claim 1, wherein said linker material is an integral component of said substrate.

8. The assay member of claim 1, wherein said linker material comprises a layer of material applied to said substrate.

9. The assay member of claim 8, wherein said layer of linker material is applied to discrete portions of said substrate, said discrete portions corresponding to said regions in which said members of said plurality of biomolecular samples are bound.

10. The assay member of claim 1, further including a coupler material interposed between said linker and said biomolecular samples so that said samples are bound to said substrate through said linker material and said coupler.

11. The assay member of claim 1 wherein the linker is selected from the group consisting of: silanes, aryl acetylene, diamines, diacids, polyalcohols, polyesters, polyethers, polylysine, polyarginine, polystyrene sulfonate, dextran sulfate, chondroiting, polyacrylic acid, polymethacrylic acid, polyacrylamide, polyallylamine, maleic acid, substituted or unsubstituted polyalkylenes, polyamines, polyamides, polysufonates, polyoxides, polyalkyleneglycols, polystyrenic based polymers, polyacetals, polysaccharides, polycarbonates, polyurethanes, polyureas, polyamides, polyethyleneimines, polyarylene sulfides, polysiloxanes, polyimides, polyacetates, polymers of monoethylenically unsaturated monomers, polymers of polyvinylidene monomers and mixtures and copolymers of the above polymers.

12. The assay member of claim 10, wherein the coupler is selected from the group consisting of: silanes, aryl acetylene, diamines, diacids, polyalcohols, polyesters, polyethers, polylysine, polyarginine, polystyrene sulfonate, dextran sulfate, chondroiting, polyacrylic acid, polymethacrylic acid, polyacrylamide, polyallylamine, maleic acid, substituted or unsubstituted polyalkylenes, polyamines, polyamides, polysufonates, polyoxides, polyalkyleneglycols, polystyrenic based polymers, polyacetals, polysaccharides, polycarbonates, polyurethanes, polyureas, polyamides, polyethyleneimines, polyarylene sulfides, polysiloxanes, polyimides, polyacetates, polymers of monoethylenically unsaturated monomers, polymers of polyvinylidene monomers and mixtures and copolymers of the above polymers.

13. The assay member of claim 1, wherein said plurality of biomolecular samples comprises at least 500 different biomolecular samples.

14. A method of manufacturing a flexible microarray assay member, said method comprising the steps of:
providing a flexible substrate having a linker material disposed upon at least a portion of a first surface of said flexible substrate, said linker material being capable of binding to a biomolecular species;
providing a plurality of biomolecular samples, each member of said plurality being of different composition from the other members of said plurality; and
coating said plurality of biomolecular samples onto said substrate so that each member of said plurality is disposed in a different region of said substrate and bound to said substrate through said linker material.

15. The method of claim 14 including the further steps of:
providing a support member, said support member having a surface which is capable of binding to the biomolecules comprising said plurality of biomolecular samples; and
contacting said substrate having said plurality bound thereto with said support member so as to transfer at least a portion of said plurality to said support member whereby there is provided an array of biomolecular samples on said support member.

16. A method of manufacturing a microarray assay member, said method comprising the steps of:
providing a flexible substrate member;
providing a plurality of biomolecular samples, each member of said plurality being of a different composition from the other members of said plurality;
coating the plurality of biomolecular samples onto said substrate so that each member of said plurality is disposed in a different region of said substrate;
providing a support member having a surface which is capable of binding to the biomolecules comprising said plurality of biomolecular samples; and
contacting said substrate having said plurality of biomolecular samples coated thereupon to said support member so as to transfer at least a portion of said plurality of biomolecular samples to said support member; whereby said samples are bound thereto.
